(19) United States
(12) Patent Application Publication
(10) Pub. No.: US 2026/0306027 A1
(43) Pub. Date: Oct. 8, 2026

(54) SYSTEMS, DEVICES, AND METHODS FOR REDUCING SIGNAL NOISE

(71) Applicant: Empatica Srl, Milano (IT)

(72) Inventors: Simone TOGNETTI, Vimodrone (IT); Giulia REGALIA, Milano (IT); Rafael CORDERO ALVAREZ, Milano (IT); Weixuan CHEN, Cambridge, MA (US)

(21) Appl. No.: 19/460,072

(22) Filed: Jan. 26, 2026

Related U.S. Application Data
(63) Continuation of application No. PCT/US2024/039838, filed on Jul. 26, 2024.
(60) Provisional application No. 63/515,829, filed on Jul. 26, 2023.

(51) Int. Cl. A61B 5/00

(52) U.S. Cl. CPC A61B 5/7203; A61B 5/725; A61B 2562/0219

(57) ABSTRACT

An apparatus includes one or more source sensors configured to measure one or more source signals, the one or more source signals including a source signal component and a source noise component and at least one reference sensor configured to measure at least one reference signal. The apparatus includes a processor operatively coupled to the one or more source sensors and the at least one reference sensor. The processor is configured to receive the one or more source signals and the at least one reference signal, process the one or more source signals using one or more adaptive filters that uses as a reference at least one reference signal from at least one reference sensors to produce a filtered output, the filtered output having less than or equal to of the source noise component than the one or more source signals.

### CROSS-REFERENCE TO RELATED APPLICATIONS

This application is a continuation of International Patent Application No. PCT/US2024/039838, titled, “SYSTEMS, DEVICES, AND METHODS FOR REDUCING SIGNAL NOISE,” filed Jul. 26, 2024, which claims priority to and benefit of U.S. Provisional Patent Application No. 63/515,829, titled, “SYSTEMS, DEVICES, AND METHODS FOR REDUCING SIGNAL NOISE,” filed Jul. 26, 2023, the disclosure of which is incorporated herein by reference in its entirety.

### TECHNICAL FIELD

Devices, systems, and methods disclosed herein relate to reducing signal noise.

### BACKGROUND

Signals from sensors, such as photoplethysmography (PPG) sensors, can often include noise associated with the sensor itself (e.g., high frequency noise, etc.) and from the movement of the sensor, such as when the sensor is a component in a wearable. Adaptive noise removal is a method for estimating the true value/nature of signals that are corrupted by additive noise or interference.

Previous methods for reducing noise have included using fix-lag Kalman smoothing, which can be computationally heavy and is not configured for use on micro-controllers. Recursive least squares (RLS) methods have also been used for reducing noise in a signal. However, RLS can have instability issues (e.g., diverging output) when operating under the effects of finite precision arithmetic, and, in particular, single-precision environments. Additionally, existing methods may not take into account the motion of a sensor, which can affect the noise. Thus, there is a need for a stable, less computationally intensive, and accurate noise reducing device, system, and/or method.

### SUMMARY

Described here are systems, devices, and methods for motion artifact removal.

In some embodiments, an apparatus includes one or more source sensors configured to measure one or more source signals. The one or more source signals includes a source signal component and a source noise component. The apparatus includes at least one reference sensor configured to measure at least one reference signal. The apparatus includes a processor operatively coupled to the one or more source sensors and the at least one reference sensor. The processor is configured to receive the one or more source signals and the at least one reference signal and to process the one or more source signals using one or more adaptive filters that uses as a reference at least one reference signal from at least one reference sensors to produce a filtered output. The filtered output has less than or equal to of the source noise component than the one or more source signals.

In some embodiments, a method includes receiving one or more source signals measured by one or more source sensors. The method includes receiving a plurality of reference signals measured by a plurality of reference sensors, the plurality of reference sensors including at least two different types of sensors. The method includes processing the one or more source signals using one or more adaptive filters that uses as a reference at least one reference signal from the plurality of reference signals to produce a filtered output, the filtered output having less than or equal to of the source noise component than the one or more source signals.

In some embodiments, a method includes receiving one or more source signals measured by one or more source sensor. The method includes receiving a plurality of reference signals measured by at least one sensor associated with a user and at least one sensor configured to measure movement. The plurality of reference sensors includes at least two different types of sensors. The method includes processing the one or more source signals using a first adaptive filter that uses as a reference at least one reference signal from the at least one sensor associated with the user to produce a first output. The method includes processing the first output using one or more adaptive filters that uses as a reference at least one reference signal from the at least one sensor configured to measure movement to produce a filtered output, the filtered output having less than or equal to of the source noise component than the one or more source signals.

### BRIEF DESCRIPTION OF THE DRAWINGS

FIG. 1A is a block diagram of a system for motion artifact removal, according to an embodiment.

FIG. 1B is a block diagram of a compute device of the system of FIG. 1A, according to an embodiment.

FIG. 2 is a block diagram of a network of systems and devices for motion artifact removal, according to an embodiment.

FIG. 3 is a schematic diagram of a sensing device, according to an embodiment.

FIG. 4 is a flow chart illustrating a method of motion artifact removal, according to an embodiment.

FIG. 5 is a diagram of a motion artifact removal workflow, according to an embodiment.

FIG. 6 is an example of implementing a motion artifact removal method, according to an embodiment.

FIG. 7 is another example of implementing a motion artifact removal method, according to an embodiment.

### DETAILED DESCRIPTION

In some embodiments, systems, apparatuses, and methods for noise reduction or removal are disclosed herein. Aspects disclosed herein adaptively remove noise from an input/source/primary signal when a reference signal and/or additional sensor data associated with the source of noise is available. For example, when the primary signal is a PPG signal associated with a user, the reference input can be a secondary PPG signal and/or additional sensor data such as accelerometer data. When input signals have noise, artifacts, and/or are generally non-stationary, aspects of the adaptive approach(es) disclosed herein can avoid undesired patterns that typically occur due to prior art approaches that fail to account for the non-stationary nature.

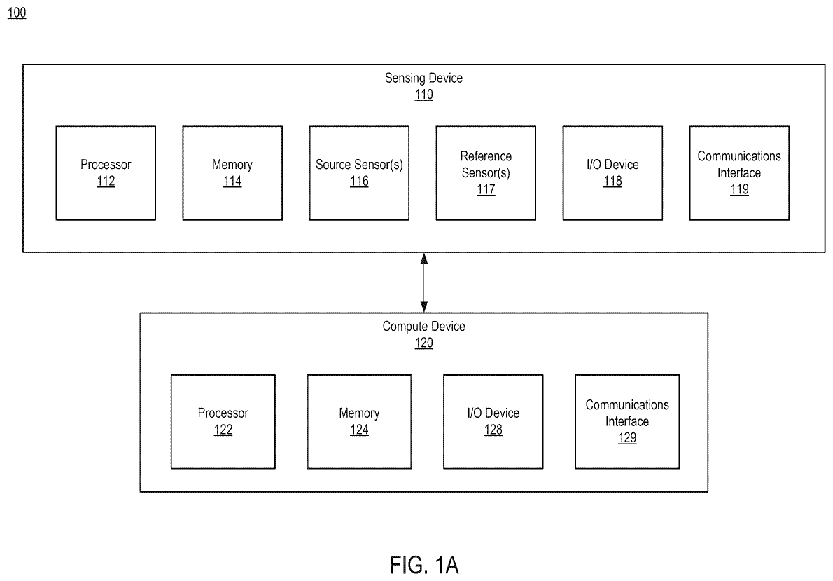

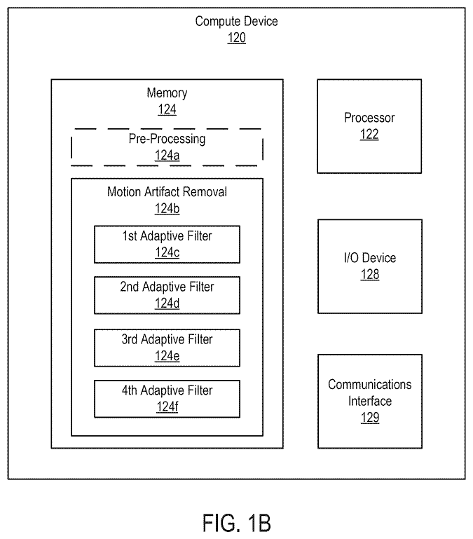

FIG. 1A is a block diagram of a system for motion artifact removal, according to an embodiment. The system can be configured to generate, receive, and process signals or data from one or more sensor(s). The system includes a sensing device 110 operably coupled to a compute device 120. In some embodiments, the sensing device 110 and the compute device 120 can be implemented as separate or different devices, which can be operatively coupled to one another. In some embodiments, the sensing device 110 and the compute device 120 can be implemented on the same device.

The sensing device 110 can be configured to collect information about a user. The sensing device 110, for example, can be, or included in, a wearable device, such as a smart watch, a sleeve, a band, or the like. In some embodiments, the sensing device 110 is configured to measure one or more characteristics or other data associated with a user and/or the sensing device 110. For example, the sensing device 110 can be configured to measure one or more of volume changes, electrical activity, cardiac vibrations, heart rate, pulse rate, blood pressure, muscle electrical potential, nerve electrical potential, temperature, brain waves, motion, measures of activity, number of steps taken, location, acceleration, pace, distance, altitude, direction, velocity, speed, time elapsed, time left, and/or the like. In some embodiments, the sensing device 110 can be configured to collect data of the user at predetermined times and/or time intervals. In some embodiments, the sensing device 110 can be configured to collect data of the user during predetermined activities (e.g., during rest and/or sleep). The sensing device 110 includes a processor 112, a memory 114, a source sensor(s) 116, reference sensor(s) 117, an input/output (I/O) device 118, and a communications interface 119 (or a multiplicity of such components), each operatively coupled to one another (e.g., via a system bus, a network, etc.).

The processor 112 can be, for example, a hardware based integrated circuit (IC), or any other suitable processing device configured to run and/or execute a set of instructions or code. For example, the processor 112 can be a general-purpose processor, a central processing unit (CPU), an accelerated processing unit (APU), an application specific integrated circuit (ASIC), a field programmable gate array (FPGA), a programmable logic array (PLA), a complex programmable logic device (CPLD), a programmable logic controller (PLC) and/or the like. The processor 112 can be operatively coupled to the memory 114 through a system bus (for example, address bus, data bus and/or control bus).

The memory 114 can be, for example, a random-access memory (RAM), a memory buffer, a hard drive, a flash memory, a read-only memory (ROM), an erasable programmable read-only memory (EPROM), and/or the like. In some instances, the memory 114 can store, for example, one or more software programs and/or code that can include instructions to cause the processor 112 to perform one or more processes, functions, and/or the like. In some implementations, the memory 114 can include extendable storage units that can be added and used incrementally. In some implementations, the memory 114 can be a portable memory (for example, a flash drive, a portable hard disk, and/or the like) that can be operatively coupled to the processor 102. In some instances, the memory 114 can be operatively coupled with the sensing device 110 and/or another compute device (e.g., compute device 120 or another compute device that is not shown). For example, a remote database device can serve as a memory and be operatively coupled to the sensing device 110 and/or compute device 120. In some embodiments, the memory 114 and processor 112 may be implemented on a single chip. In other embodiments, they can be implemented on separate chips.

The source sensor(s) 116 can include one or more sensor(s) configured to measure a characteristic (e.g., PPG, electrodermal activity (EDA), blood pressure, heart rate, skin temperature, etc.) of a user and generate a source signal. The source sensor(s) 116 can include a PPG sensor, an EDA sensor, an accelerometer, a skin temperature sensor, an ambient temperature sensor, a gyroscope, a global positioning system (GPS) sensor, an electrical sensor, a conductance sensor, a magnetometer, a capacitive sensor, an optical sensor, a barometer sensor, a respiration sensor, a blood pressure sensor, a humidity sensor, a camera, and/or the like. In an embodiment, the source sensor(s) 116 can be a PPG sensor for capturing PPG information. In some embodiments, the source sensor(s) 116 can be a green PPG sensor, i.e., a PPG sensor configured to operate with green or near green wavelengths of light. Alternatively, the source sensor(s) 116 can be a red PPG sensor, i.e., PPG sensor configured to operate with red, infrared, or other near red wavelengths of light. In some embodiments, the source sensor(s) 116 can be configured to operate with any wavelength of light. In some embodiments, the data from the source sensor(s) 116 is stored in the memory 114. In some embodiments, the processor 112 can be configured to control the operation of the source sensor(s) 116. For example, the processor 112 can be configured to activate the source sensor(s) 116 and/or change one or more operational parameters (e.g., light wavelengths, length intensity, sampling frequency, etc.) of the source sensor(s) 116. The source sensor(s) 116 can be configured to operate continuously, sporadically, and/or periodically.

The reference sensor(s) 117 can include one or more sensor(s) configured to measure characteristics of the user and/or the sensing device 110 and generate one or more reference signals. The measurements of the reference sensor(s) can be used to process a source signal from the source sensor(s) 116, e.g., to decrease noise in the source signal. The reference sensor(s) 117 can include the same type, similar, and/or complementary sensor(s) as the source sensor(s) 116 and/or other types of sensors. For example, the reference sensor(s) 117 can include a PPG sensor, an EDA sensor, an accelerometer, a skin temperature sensor, an ambient temperature sensor, a gyroscope, a global positioning system (GPS) sensor, an electrical sensor, a conductance sensor, a magnetometer, a capacitive sensor, an optical sensor, a barometer sensor, a respiration sensor, a blood pressure sensor, a humidity sensor, a camera, and/or the like. In some embodiments, the reference sensor(s) 117 can include a 3-axis accelerometer and/or a 3-axis gyroscope configured to measure one or more of movement and/or position. In some embodiments, the source sensor(s) 116 can include a green PPG sensor and the reference sensor(s) 117 can include a red PPG sensor and/or an accelerometer. In some embodiments, the source sensor(s) 116 and the reference sensor(s) 117 can be the same type of sensor (e.g., PPG sensor) but operate under different conditions (e.g., at different wavelengths, at different locations or distances relative to a skin surface, at different intensities of light, etc.).

In some embodiments, parameters (e.g., sample rate, power, etc.) associated with the source sensor(s) 116 and/or the reference sensor(s) 117 can be adjusted during operation. In some embodiments, the source sensor(s) 116 and/or the reference sensor(s) 117 can be configured to operate intermittently. For example, the source sensor(s) 116 and/or the reference sensor(s) 117 can be intermittently turned off to save power (e.g., battery power) during operation.

The I/O device 118 can include an input device and/or an output device, such as, for example, a display (e.g., Cathode Ray tube (CRT) display, Liquid Crystal Display (LCD), Light Emitting Diode (LED) display, Organic Light Emitting Diode (OLED) display, and/or the like), mouse, keyboard, microphone, touch screen, speaker, scanner, headset, printer, camera, and/or the like. For example, the I/O device 118 may include an input device for a user to input commands and/or an output device for a user to receive output (e.g., PPG readings, electrodermal activity readings, etc. on a display device). In some embodiments, the I/O device 118 can be used as a source sensor(s) to provide alerts to a user, e.g., to indicate to a user that there is too much movement for sensor data capture. In some embodiments, the I/O device 118 can instruct a user to perform certain activities (e.g., to lay down or to minimize movement), e.g., to facilitate cleaner data capture by sensor(s). In some embodiments, the I/O device 118 can display information received from the compute device 120.

The communications interface 119 of the sensing device 110 can be configured to receive information and/or send information to other devices (e.g., sensing device 110). The communications interface 119 can be a wired or wireless communications interface. The communications interface 119 can, for example, be configured to send information from the source sensor(s) 116 and/or the reference sensor(s) 117 to the compute device 120. In some embodiments, the communications interface 119 can receive data, signals, and/or instructions from the compute device 120 The compute device 120 can be configured to process and/or analyze sensor data, e.g., received from the sensing device 110, and/or other data, e.g., received from a user, a database, or other source. For example, the compute device 120 can be configured to filter, rectify, differentiate, integrate, enhance, pre-process, combine the sensor data. In some embodiments, the compute device 120 can be nearby the sensing device 110, such as, for example, a local computer, laptop, mobile device, tablet, etc. In some embodiments, the compute device 120 can be a server that is remote from the sensing device 110 but can communicate with the sensing device 110, e.g., via a network. In some embodiments, the sensing device 110 can be configured to transmit sensor data to a nearby device (e.g., a user device such as a mobile device) via a wireless network (e.g., Wi-Fi, Bluetooth, etc.), and then that device can be configured to transmit the sensor data to the compute device 120 for further processing and/or analysis. In some embodiments, the compute device 120 is implemented as or includes a user device.

The compute device 120 can include a processor 122, a memory 124, an I/O device 128, and a communications interface 128 (or a multiplicity of such components). The memory 124 can be, for example, a random access memory (RAM), a memory buffer, a hard drive, a flash memory, a database, an erasable programmable read-only memory (EPROM), an electrically erasable read-only memory (EEPROM), a read-only memory (ROM), and/or so forth. In some embodiments, the memory 124 stores instructions that cause processor 122 to execute modules, processes, and/or functions associated with processing and/or analyzing sensor data from sensing device 110. In some embodiments, the memory 124 stores information associated with more than one user. For example, compute device 120 can be a household account, a medical provider account, and/or the like, and the memory 124 can be configured to store information associated with one or more users associated with that account. The administrator account can be utilized to allow one or more users (e.g., healthcare professionals, caretakers, etc.) to access information during operation.

The processor 122 of compute device 120 can be any suitable processing device configured to run and/or execute functions associated with processing and/or analyzing sensor data from the sensing device 110. The processor 122 can be a general purpose processor, microcontroller, a Field Programmable Gate Array (FPGA), an Application Specific Integrated Circuit (ASIC), a Digital Signal Processor (DSP), and/or the like. In some embodiments, the processor 122 and the processor 112 are the same processor. In some embodiments, the processor 122 can be configured to process a source signal from the source sensor(s) 116 and/or other sensed information, e.g., to reduce signal noise in the source signal and/or to extract certain information from the source signal. The sensed information can include, for example, one or more of raw sensor signal information, processed sensor signal information, timestamp information, time window information, contextual information, and/or the like. In some embodiments, the sensed information can indicate a time period during which the sensed information was obtained/collected. In some embodiments, source sensor(s) the processor 122 can be configured to send instructions to the sensing device 110 to cause the sensing device 110 to operate according to one or more parameters. For example, the processor 122 can be configured to send instructions to the sensing device 110 to take measurements at predetermined times and/or intervals.

The source signal can include a source noise component. In some embodiments, the source noise component can include noise from multiple sources. The processor 122 can also receive one or more reference signals from the reference sensor(s) 117 and/or other sensed information. The one or more reference signals can each include a reference noise component. The reference noise component can be correlated with a noise source, e.g., motion. The reference noise component can be associated with the source noise component. In particular, the source signal and the reference signal can be collected during the same time period such that the reference noise component can be used to identify (e.g., isolate or approximate) the source noise component.

In some embodiments, the processor 122 can be configured to filter the source signal and/or the reference signal(s). Filtering the signals can include removing artifacts. For example, removing artifacts can be based on a predetermined bandwidth and removing all artifacts that are not in the predetermined bandwidth. In some embodiments, the filter can include a band-pass filter, a high-pass filter, and/or other type of filter. The type of filter applied by the processor 122 can depend on the type of source data being collected. For example, when the source signal is a PPG signal, the processor 122 can be configured to apply a filtering algorithm that removes frequencies of data outside of those commonly associated with heart rate (e.g., 24-240 beats/minute).

In some embodiments, the processor 122 can be configured to process the source signal using a motion artifact removal (MAR) algorithm. The MAR algorithm can implement adaptive noise removal or cancellation to reduce the amount of noise in the source signal. In some embodiments, the MAR algorithm is configured to use as reference signal(s) the reference signal(s) captured by the one or more reference sensor(s) 117, e.g., to remove artifacts from the source signal. In some embodiments, the MAR algorithm can process the source signal using one or more QR-RLS algorithm(s) or QR decomposition-based recursive least squares algorithm(s). The QR-RLS algorithm(s) can be adaptive in that the algorithm(s) recursively adapt one or more coefficients (e.g., based on one or more reference signal(s)) to reduce the cost of a weighted linear least squares cost function associated with the signals that are input into the QR-RLS algorithm(s). Other algorithms can also be utilized. For example, other adaptive filters that can adapt coefficients using one or more reference signal(s) correlated to noise can be used. For example, least-mean-square (LMS), normalized LMS (NLMS), other recursive least squares (RLS) algorithms, and/or the like can be utilized.

In some embodiments, the MAR algorithm can include a cascade of filters, where each filter uses a different reference signal having a different noise component. For example, if the source signal is from a first PPG sensor (e.g., a green PPG sensor), a first filter can utilize a second PPG sensor signal (e.g., red PPG sensor signal) as a reference to filter the source PPG signal, a second filter can utilize an x-axis accelerometer signal as a reference to filter an output of the first filter, a third filter can utilize a y-axis accelerometer signal as a reference to filter an output of the second filter, and a fourth filter can utilize a z-axis accelerometer signal as a reference to filter an output of the third filter. In some embodiments, the output of the MAR algorithm can be post-processed by the processor 122. Post-processing can include changing the format of the output (e.g., into a user readable format), identifying anomalies in the output, and/or the like. Post-processing can include smoothing the output signal (e.g., via averaging) and/or removing residual baseline wander (e.g., applying a band-pass filter). In some embodiments, post-processing can be replaced by a simple smoother such as a low-pass filter and/or the like.

In some embodiments, the processor 122 can be configured to implement other adaptive filtering algorithms. Suitable examples of other adaptive filtering algorithms are described in U.S. Pat. No. 10,134,378, titled “SYSTEMS, APPARATUSES AND METHODS FOR ADAPTIVE NOISE REDUCTION,” issued on Nov. 20, 2018.

In some embodiments, the processor 122 can be configured to generate and present an output to the user. The output can include the processed signal in a format that is understandable by the user. In some embodiments, the processor 122 can provide an output after the filtering to another process (e.g., implemented by the processor 122 and/or another processor operatively coupled to the processor 122) for further analysis. For example, the cleaned output after applying the filtering can be provided to a heart or pulse rate process and/or other process for evaluating one or more physiological characteristics and/or conditions of a user. While the processor 112 and the processor 122 are described with specific functions above, it can be appreciated that the processor 112 of the sensing device 110 can be configured to execute some or all of the processes the processor 122 and vice versa.

The I/O device 128 of the compute device 120 can be similar to the I/O device 118 of the sensing device 110. For example, the I/O device 128 can include an input device for receiving one or more inputs and/or commands from a user and/or an output device for presenting information to a user. The I/O device 128 can include any type of peripherals, such as an input device, an output device, a mouse, keyboard, microphone, touch screen, speaker, scanner, headset, printer, camera, and/or the like. In some embodiments, the I/O device 128 can be used by a user to view the processed data. For example, if the system processes PPG or heart rate data, the I/O device 128 can be utilized to display heart rate metrics derived from the PPG or pulse rate data to the user.

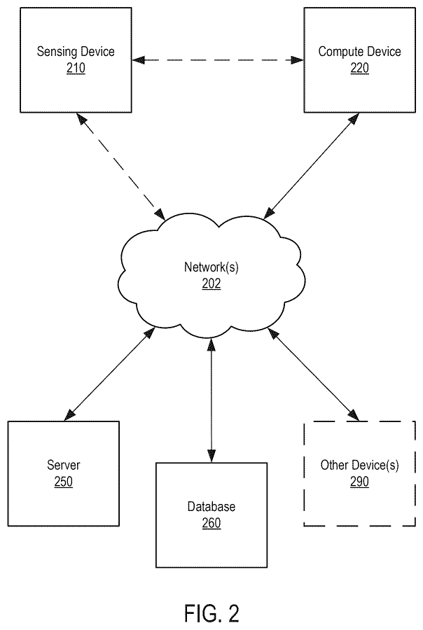

The communications interface 129 of the compute device 120 can be configured to receive information and/or send information to other devices (e.g., sensing device 110, and/or other compute devices as depicted in FIG. 2). The communications interface 128 can be a wired or wireless communications interface. In some embodiments, the communications interface 129 can be configured to receive data from the sensing device 110, including the data associated with the source sensor(s) 116 and/or the reference sensor(s) 117.

FIG. 1B provides a more detailed view of the compute device 120. As described above, the compute device 120 can be configured to process or adaptively filter the source signal(s) from the source sensor(s) 116, e.g., using one or more adaptive filters and reference signal(s) including noise components that are corelated with those of the source signal(s).

The memory 124 can store instruments that can cause processor 122 to execute or implement modules, processes, and/or functions, illustrated as pre-processing 124a and motion artifact removal 124b. The pre-processing 124a can be optional. In some embodiments where pre-processing 124a is implemented, it can include smoothing, e.g., through low-pass filtering, band-pass filtering, spike noise correction, and/or other types of filtering and/or averaging. In some embodiments, pre-processing 124a can first include spike noise correction, source signal(s) selection and/or combination, and then filtering and/or averaging. In some embodiments, multiple PPG source sensor signals are combined to get a single source signal. In some embodiments, motion artifacts are removed to multiple PPG source signals and the outputs are combined to a single source signal. The pre-processing 124a can remove one or more artifacts from a source signal. For example, the pre-processing 124a can include applying a band-pass filter that removes artifacts outside of a predetermined bandwidth of frequencies. The specific type of filtering or smoothing that is applied can be passed on the type of source signal and/or reference signal. For example, if the source signal is a PPG signal, then the predetermined bandwidth may correspond a range of heart beats per minute.

After pre-processing 124a, or without pre-processing 124a in cases where it is not implemented, the motion artifact removal algorithm or MAR 124b can be implemented. In some embodiments the MAR 124b includes a one-step filtering process (e.g., red PPG based filter), a three-step filtering process (e.g., 3-axis accelerometer based filters), or a four-step filtering process (e.g., red PPG based filter and 3-axis accelerometer based filters). The MAR 124b can include one or more adaptive filters, including, a first adaptive filter 124c, a second adaptive filter 124d, a third adaptive filter 124e, and a fourth adaptive filter 124f. While four adaptive filters are depicted in FIG. 1B, it can be appreciated that the MAR 124b can include additional and/or fewer adaptive filters. The adaptive filters 124c, 124d, 124e, 124f can be any type of adaptive filter capable of reducing signal noise. For example, the adaptive filters 124c, 124d, 124e, 124f can include a Kalman filter (e.g., a fixed lag Kalman smoother), a least means squares (LMS) filter, a normalized least means squares (MLMS) filter, a recursive least squares (RLS) filter, and/or a QR-RLS filter. In some embodiments, each adaptive filter 124c, 124d, 124e, 124f can be the same type of filter. For example, each adaptive filter 124c, 124d, 124e, 124f can be a QR-RLS filter. Alternatively, one or more of the adaptive filters 124c, 124d, 124e, 124f can be different from each other.

In some embodiments, the adaptive filters 124c, 124d, 124e, 124f can be cascading, where the output of a first filter is provided as the input into a second filter, and so on and so forth. For example, the first adaptive filter 124c can receive as an input the pre-processed signal from 124a and generate a first output, the second adaptive filter 124d can receive as an input the first output and generate a second output, the third adaptive filter 124e can receive as an input the second output and generate a third output, and the fourth adaptive filter 124f can receive as an input the third output and generate a final output. In some embodiments, the order of the adaptive filters 124c, 124d, 124e, 124f of the MAR 124b can be varied or certain adaptive filters 124c, 124d, 124e, 124f can be omitted, e.g., depending on the source signal(s) and/or reference signal(s).

While not depicted, in some embodiments, the memory 124 may store additional instructions associated with post-processing the output of the MAR 124b. For example, further smoothing, averaging, and/or feature extraction can be performed after processing the source signal(s) with MAR 124b.

In some embodiments, the adaptive filters 124c, 124d, 124e, 124f can have a first input or primary input that is the source signal (e.g., source signal captured by source sensor(s) 116), or a pre-processed source signal, and a second input or reference input that is a reference signal, or a pre-processed reference signal. As described above, the reference signal can be a signal that includes a noise component that is correlated with the noise component of a source signal. As such, reference signal can be used to isolate the noise component in the source signal. In some embodiments, each adaptive filter 124c, 124d, 124e, 124f can use a different reference signal. For example, when the source signal is a PPG signal (e.g., a green PPG signal), the first adaptive filter 124c may use a different PPG signal (e.g., a red PPG signal) as its reference, the second adaptive filter 124d may use an x-axis accelerometer signal as its reference, the third adaptive filter may use a y-axis accelerometer signal as its reference, and the fourth adaptive filter may use a z-axis accelerometer signal as its reference.

The reference signals can be used to identify or isolate a noise component associated with the source signal during micro-motion (e.g., low intensity movements), such as typing and the like, and macro-motion (e.g., high intensity movements), such as gesturing, arm sweeping, and the like. When the source signal is a PPG signal, the use of another PPG signal as a reference can be effective for filtering out noise components attributable to micro-motion and macro-motion. The use of accelerometer signals (e.g., 3-axis accelerometer signals) can be effective for filtering out noise components attributable to macro-motions and locomotion. As such, when the source signal is a PPG signal, the use of both PPG signals and 3-axis accelerometer signals allows for a variety of motion artifacts to be filtered out of the source signal.

In an example embodiment, the compute device 120 can receive a source signal from the source sensor(s) 116 and a plurality of reference signals from a plurality of reference sensors 117. The source signal can be a PPG signal captured by a green PPG sensor, and the reference signals can include a PPG signal captured by a red PPG sensor and x-, y-, and z-axis acceleration signals captured by one or more accelerometers. The source PPG signal can be pre-processed, e.g., via pre-processing 124a. For example, a bandpass filter can be applied to the source PPG to remove artifacts. The MAR algorithm 124b can then be applied to the source PPG signal. In particular, the first adaptive filter 124c implemented as a QR-RLS filter can receive as a primary input the source PPG signal and as a secondary or reference input the reference PPG signal and generate a first output. The second adaptive filter 124d implemented as a QR-RLS filter can receive as a primary input the first output and as a secondary or reference input the x-axis acceleration signal and generate a second output. The third adaptive filter 124e implemented as a QR-RLS filter can receive as a primary input the second output and as a secondary or reference input the y-axis acceleration signal and generate a third output. And the fourth adaptive filter 124f implemented as a QR-RLS filter can receive as a primary input the third output and as a secondary or reference input the z-axis acceleration signal and generate a fourth output. The fourth output can be post-processed, e.g., to further refine and/or smooth the fourth output to produce a final output. The final output can represent the source signal with the noise component of the source signal being substantially reduced.

FIG. 2 is a block diagram of a network including systems and devices for MAR, according to an embodiment. Such systems and devices can be configured to process source signals to remove noise, e.g., due to motion. In some embodiments, the systems and devices for MAR can include a sensing device 210 (e.g., functionally and/or structurally similar to the sensing device 110 of FIG. 1A) and/or a compute device 220 (e.g., functionally and/or structurally similar to the compute device 120 of FIG. 1A and/or compute device 220 of FIG. 2). The sensing device 210 and/or the compute device 220 can be operatively coupled to one or more other compute devices, including, for example, a server 250, a database 260, and/or one or more other device(s) 290, via one or more network(s) 202. In some embodiments, the systems and devices for MAR can optionally include other device(s) 290, such as additional sensing device(s) (e.g., functionally and/or structurally similar to the sensing device 110 of FIG. 1A) and/or additional compute device(s) (e.g., functionally and/or structurally similar to the compute device 120 of FIG. 1A and/or compute device 220 of FIG. 2).

The network 202 can be any type of network implemented as a wired network and/or wireless network and used to operatively couple the sensing device 210, the compute device 220, the server 250, the database 260, and/or other device(s) 290. The communication may or may not be encrypted. A wireless network may refer to any type of digital network that is not connected by cables of any kind. Examples of wireless communication in a wireless network include, but are not limited to cellular, radio, satellite, and microwave communication. However, a wireless network may connect to a wired network in order to interface with the Internet, other carrier voice and data networks, business networks, and personal networks. A wired network is typically carried over copper twisted pair, coaxial cable and/or fiber optic cables. There are many different types of wired networks including wide area networks (WAN), metropolitan area networks (MAN), local area networks (LAN), Internet area networks (IAN), campus area networks (CAN), global area networks (GAN), like the Internet, and virtual private networks (VPN).

The network 202 may include or be coupled to the server 250 and the database 260 for processing and/or storage. The database 260 can be any device configured to store data from other components of system. For example, the database 260 can include instructions for processing signals, signal data, processed signal data, signal repositories, and/or the like. In some embodiments, the database 260 can include the final outputs from processing the source signals (e.g., signals captured by source sensor(s) 116 of a sensing device 110). The server 250 can be any device configured to process signals and/or data received from the sensing device 210 and/or the compute device 220. In some embodiments, the server 250 can be configured to execute some of the processes of the sensing device 210 and/or the compute device 220.

Similar to other sensing devices described above, the sensing device 210 can be operatively coupled to the compute device 220. For example, the sensing device 210 can be operatively coupled to the compute device 220 via near-field communication, a wireless connection (e.g., Wi-Fi, Bluetooth, etc.), and/or a wired connection. Optionally, the sensing device 210 can be coupled to the network(s) 202 and/or other compute devices (e.g., server 250, database 260, other device(s) 290). The sensing device 210 can be operatively coupled to the compute device 220 and/or one or more other compute devices such that the sensing device 210 can send information (e.g., sensor signals) to and/or receive information (e.g., instructions for monitoring a patient or subject, parameters for operation, etc.) from one or more such devices.

In some embodiments, the other device(s) 290 can include (e.g., functionally and/or structurally similar to the sensing device 110 of FIG. 1A) and/or additional compute device(s) (e.g., functionally and/or structurally similar to the compute device 120 of FIG. 1A and/or compute device 220 of FIG. 2). In some embodiments, the other device(s) 290 can include compute devices that are associated with one or more third-parties, such as, for example, an administrator, a physician or healthcare provider, a hospital, a caretaker, etc.

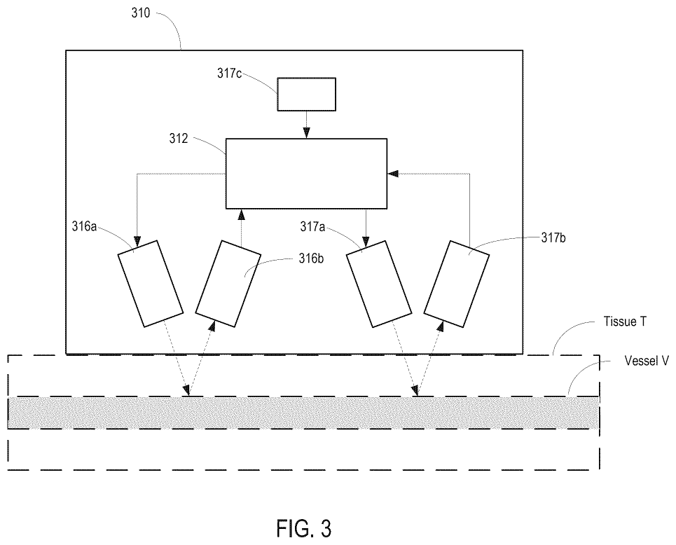

FIG. 3 is a schematic diagram of a sensing device 310 (e.g., structurally and/or functionally similar to the sensing device 110 of FIG. 1A and/or the sensing device 210 of FIG. 2), according to an embodiment. The sensing device 310 can include one or more PPG sensors and/or other sensors. In an embodiment, the sensing device 310 can be configured to measure a pulse rate associated with a vessel V of a subject (e.g., a user, patient, etc.). In some embodiments, the vessel V includes one vessel. In some embodiments, the vessel V includes more than one vessel. As depicted in FIG. 3, the sensing device 310 can be placed near the vessel V, e.g., adjacent to and/or engaged with a surface of tissue T near the vessel V. More specifically, the sensing device 310 can be placed against a skin surface that is above the vessel V.

The sensing device 310 includes a processor 312 (e.g., structurally and/or functionally similar to the processor 112 of FIG. 1A), a source sensor (e.g., functionally and/or structurally similar to the source sensor(s) 110 of FIG. 1A), and reference sensors (e.g., functionally and/or structurally similar to the reference sensor 117 of FIG. 1A). The source sensor can include a first PPG sensor (also referred to herein as a source PPG sensor) including an electromagnetic radiation (EMR) or light emitter 316a and a corresponding EMR or light detector or receiver 316b. The reference sensors can include a second PPG sensor (also referred to herein as a reference PPG sensor) including an EMR/light emitter 317a and an EMR/light detector or receiver 317b and an accelerometer 317c. The source and reference sensors 316a/316b, 317a/317b, and 317c can be operatively or communicatively coupled to the processor 312. In some embodiments, the reference sensors can include a combination of any number of sensors. In some embodiments, each sensor of the reference sensors can flash different wavelengths of light sequentially to obtain additional sensor data.

The source PPG sensor can be configured to measure a PPG waveform or signal of the subject. In particular, the emitter 316a is configured to direct EMR or light toward the vessel V, and the detector 316b is configured to measure EMR or light, including EMR or light reflected by the vessel V. The EMR or light signal measured by the detector 316b can include a blood pulse reflection (BPR) component and a noise component (e.g., due to ambient light and/or other environmental conditions). The emitter 316a and the detector 316b can be configured to function with specific intensities and/or wavelengths of light. For example, the emitter 316a can be configured to emit any wavelength of light that is safe and suitable for capture the pulsatile movement of the vessel V, and the detector 316b can be configured to detect the wavelength of light emitted by the emitter 316a (or a range of wavelengths including the wavelength of light emitted by the emitter 316a). In an example embodiment, the source PPG sensor can be a green PPG sensor, and as such, the emitter 316a can be configured to emit a green or near green wavelength of light and the detector 316b can be configured to detect a green or near green wavelength of light.

In some embodiments, the processor 312 can be configured to control the operation of the emitter 316a and/or the detector 316b. For example, the processor 312 can control the wavelength and/or intensity of the output of the emitter 316a, the time of operation of the emitter 316a, and/or the like. Additionally, the processor 312 can receive the source signal measured by the detector 316b. In some embodiments, the processor 312 can be configured to pre-process the source signal and/or to package the source signal for sending to a compute device (e.g., compute device 120, 220).

Similarly, the second PPG sensor can also be configured to measure a PPG waveform or signal of the subject. In particular, the emitter 317a is configured to direct EMR or light toward the vessel V, and the detector 317b is configured to measure EMR or light, including EMR or light reflected by the vessel V. The signal measured by the detector 317b can be a reference signal that includes a BPR component and a noise component. When the source PPG sensor (including emitter-detector pair 316a, 316b) and the reference PPG sensor (including emitter-detector pair 317a, 317b) operate substantially at the same time, the noise component of the reference PPG signal can be correlated with the noise component of the source PPG signal. In some embodiments, the processor 312 can control the operation of the emitter 317a and/or detector 317b. In some embodiments, the processor 312 can be configured to receive the reference signal measured by the detector 317b and, optionally, to pre-process and/or package the signal for sending to a compute device (e.g., compute device 120, 220).

The accelerometer 317c is configured to measure acceleration or movement data relating to the sensing device 310 and/or subject. For example, the accelerometer 317c can be configured to measure acceleration data in one or more axes, e.g., a x-axis, a y-axis, and/or a z-axis in a reference coordinate frame. In some embodiments, the processor 312 can be configured to control the operation of the accelerometer 317c, such as, for example, activation of the accelerometer. The measurements of the accelerometer 317c can be received by the processor 312. In some embodiments, the processor 312 can process the accelerometer 317c data. For example, the processor 312 can compute a magnitude of acceleration. In some embodiments, the processor 312 can receive and process other data such as gyroscope data.

As described above, the processor 312 can be configured to send the signals received from the source and reference sensors to a compute device (e.g., compute device 120, 220), e.g., via a communications interface (e.g., communications interface 119). The compute device can be configured to process the signals from the source and reference sensors. In particular, the compute device can be configured to process the source sensor signal to remove motion artifacts using one or more signals from the reference sensors. Alternatively, or additionally, the processor 312 can be configured to process the signals from the source and reference sensors, e.g., to remove motion artifacts or to perform a portion of the processing associated with MAR.

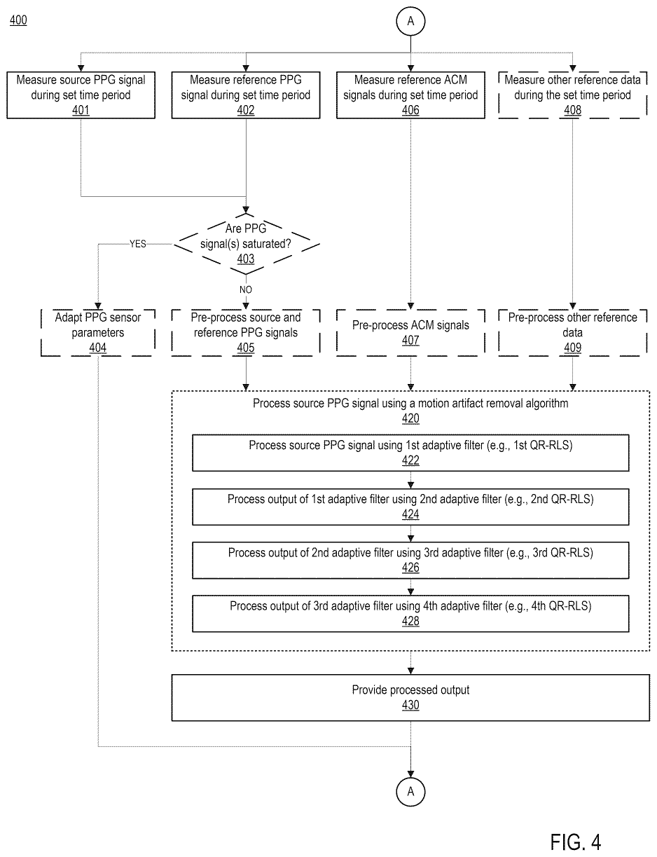

FIG. 4 is a flow chart illustrating a method 400 of implementing MAR, according to an embodiment. The method 400 can be executed by any of the systems and devices described herein, such as, for example, system 100 of FIG. 1A, the system of FIG. 2, and/or one or more compute devices or sensing devices described in FIGS. 1A-3. While the method 400 is described with reference to PPG signals and processing PPG signals, it can be appreciated that the method 400 can be applied to reducing noise and artifacts in other types of signals and/or data.

At 401, the method 400 includes measuring the source PPG signal during a set time period (e.g., 1 second, 5 seconds, 30 seconds, 1 minute, 5 minutes, 30 minutes, 1 hour, etc.). The source PPG signal can be measured by a sensor, such as, for example, any of the source sensors described herein. In some embodiments, the source PPG signal is measured using a green PPG sensor. In some embodiments, the set time period can be set and/or adjusted by a user. In some embodiments, the duration of the set time period can depend on the type of data being measured, the current state or activity level of the user, the time of day, etc. In some embodiments, the duration of the set time period can be selected to reduce the risk of having erroneous or anomalous readings. Simultaneously to 401, at 402, the method 400 includes measuring a reference PPG signal during the same set time period. The reference PPG signal can be measured by a sensor, such as, for example, any of the reference sensor(s) described herein. In some embodiments, the reference PPG signal can be measured using a different sensor than the sensor that is used to measure the source PPG signal. For example, the source PPG signal can be measured using a green PPG sensor, and the reference PPG signal can be measured using a red PPG sensor, or vice versa. Optionally, the method 400 includes determining whether one or both of the source PPG signal or reference PPG signal are saturated, at 403. If one or both PPG signals are saturated (403: YES), then optionally adapt PPG sensor parameters at 404. In some embodiments, the position of the sensors or sensing device can alternatively be adjusted such that subsequent signals measured by the sensors are not saturated. In some embodiments, the sensors can continue to take measurements until they capture signals that are not saturated and therefore can be processed using the MAR algorithm. In some embodiments, a sensing device and/or compute device (such as any of the ones described herein) may implement methods for compensating for the saturation. For example, the sensing device and/or compute device may adjust the operation of the PPG sensors (e.g., by decreasing or increasing an intensity of the light/EM and/or adjusting a position of the light/EM emitters and/or receivers). Alternatively, the sensing device and/or compute device may adjust a gain of an amplifier, introduce a compensation voltage and/or current, or implement a compensation algorithm (e.g., by determining a baseline or offset). If the source and reference PPG signals are not saturated (403: NO), then the source PPG signal and/or the reference PPG signal can optionally be pre-processed, at 405, e.g., using a pre-processing algorithm such as that described with reference to pre-processing 124a of FIG. 1B. For example, the source PPG signal and/or the reference PPG signal can be filtered using a bandpass filter. In some embodiments, spikes and/or artifacts can be cancelled during 405. In some embodiments, pre-processing can include PPG source signal(s) selection and/or combination. In some embodiments, pre-processing can first include spike noise correction, source signal(s) selection and/or combination, and finally filtering and/or averaging of the signal. In some embodiments, the bandpass filter can have a bandwidth that is selected to correspond to frequencies that are commonly associated with heartbeat (e.g., between 24 beats per minute and 240 beats per minute). Such a bandpass filter can remove artifacts that are outside of the heartbeat range, such as, for example, those introduced by breathing or other motion.

At 406, the method 400 includes measuring accelerometer data during the set time period. The accelerometer data can be measured by a sensor, such as, for example, any of the reference sensor(s) described herein, including, for example, accelerometer 317c. In some embodiments, the accelerometer data include an x-axis acceleration signal, a y-axis acceleration signal, a z-axis acceleration signal, and/or acceleration signals along other axes. Optionally, the accelerometer data can be pre-processed (e.g., using a bandpass filter), at 407. In some embodiments, other reference data can also be collected during the set time period, at 408. For example, gyroscope data, magnetometer data, location data, pressure data, electrodermal activity data, and/or additional PPG or accelerometer data. Optionally, such data can also be pre-processed (e.g., using a bandpass filter), at 409.

At 420, the method 400 includes processing the source PPG signal using a MAR algorithm. In some embodiments, the MAR algorithm includes implementing a plurality of filter modules or algorithms (e.g., first adaptive filter 124c, second adaptive filter 124d, third adaptive filter 124e, and fourth adaptive filter 124f) in a cascade, where each filter module uses a different noise reference signal (e.g., the reference PPG signal, the accelerometer signals, and/or other reference signals).

In particular, the MAR algorithm includes processing the source PPG data using a first adaptive filter (e.g., first adaptive filter 124c), at 422. In some embodiments, the first adaptive filter is implemented as a first QR-RLS module or algorithm. The source PPG signal can be provided as an input into the first QR-RLS module, and the first QR-RLS module can process the source PPG signal using one of the reference signals as a reference (e.g., the reference PPG signal). The output of the first QR-RLS module can then be passed to a second adaptive filter (e.g., second adaptive filter 124d). In some embodiments, the second adaptive filter is implemented as a second QR-RLS module. At 424, the second QR-RLS module can process the output of the first QR-RLS module using another reference signal as a reference (e.g., the x-axis acceleration signal). The output of the second QR-RLS module can then be passed to a third adaptive filter (e.g., third adaptive filter 124e). In some embodiments, the third adaptive filter is implemented as a third QR-RLS module. At 426, the third QR-RLS module can process the output of the second QR-RLS module using yet another reference signal as a reference (e.g., the y-axis acceleration signal). The output of the third QR-RLS module can then be passed to a fourth adaptive filter (e.g., fourth adaptive filter 124f). In some embodiments, the fourth adaptive filter is implemented as a fourth QR-RLS module. At 428, the fourth QR-RLS module can process the output of the third QR-RLS module using yet another reference signal as a reference (e.g., the z-axis acceleration signal).

While the MAR algorithm is described as four processing steps in FIG. 4, it can be appreciated that the MAR algorithm can include fewer or additional processing steps. For example, the MAR algorithm may process the source PPG signal with one, two, three, four, five, six, seven, eight, nine, ten, or more adaptive filters. In some embodiments, each adaptive filter utilizes a different reference signal.

At 430, the method 400 includes providing a processed output. In some embodiments, the processed output can correspond to the output of the MAR algorithm. In some embodiments, the output of the MAR algorithm may be processed further (e.g., using post-processing algorithms), and the processed output may correspond to the processed output of the MAR algorithm. The processed output may be the source PPG signal with artifacts and other signal noise removed. In some embodiments, the method 400 may repeat or multiple instances of the method 400 may be implemented together. In some embodiments, the method 400 may operate continuously in real-time.

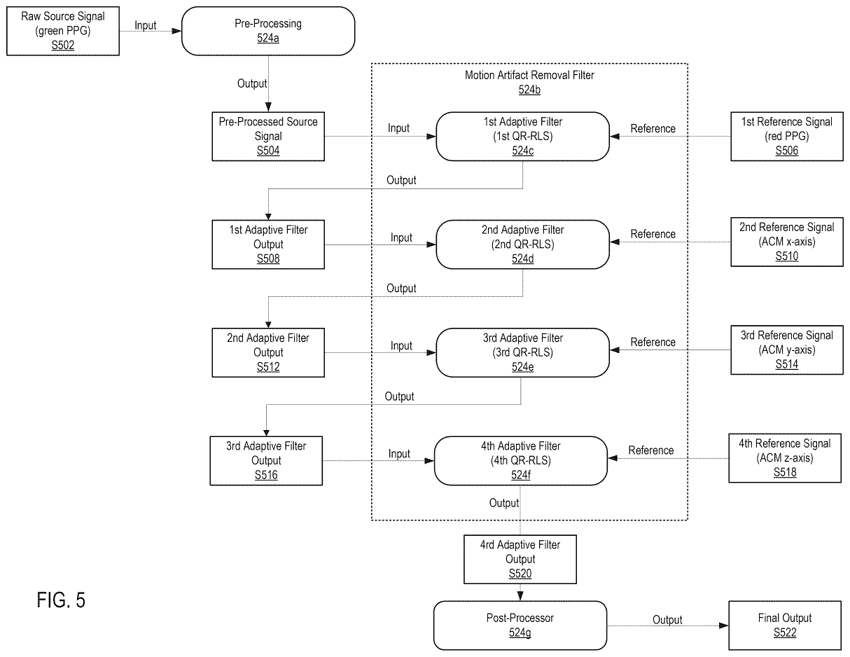

FIG. 5 is a diagram of an adaptive noise reduction workflow, which shows a different view of a method for processing a source PPG signal, according to embodiment. The workflow can be executed by any of the systems and devices described herein, such as, for example, system 100 of FIG. 1A, the system of FIG. 2, and/or one or more compute devices or sensing devices described in FIGS. 1A-3. The workflow shown in FIG. 5 can involve similar elements as those of the method 400 described with reference to FIG. 4. As such, certain details of the workflow are not described herein again.

As shown in FIG. 5, the data processing workflow starts with the source signal S502 (e.g., a green PPG signal) being filtered by the filter 524a and output as the filtered source signal S504. In some embodiments, the raw source signal is chosen based on a criterion (e.g., time of day, type of signal desired, etc.). A first adaptive filter 524c (e.g., functionally and/or structurally similar to the first adaptive filter 124c of FIG. 1B) receives the filtered source signal S504 as an input and the first reference signal S506 (e.g., a red PPG signal) as a reference input. The first adaptive filter 524c processes the filtered source signal S504 to generate the first adaptive filter output S508. A second adaptive filter 524d (e.g., functionally and/or structurally similar to the second adaptive filter 124d of FIG. 1B) receives the first adaptive filter output S508 as an input and the second reference signal S506 as a reference input. The second adaptive filter 524d processes the first adaptive filter output S508 to generate the second adaptive filter output S512. A third adaptive filter 524e (e.g., functionally and/or structurally similar to the third adaptive filter 124e of FIG. 1B) receives the second adaptive filter output S512 as an input and the third reference signal S514 as a reference input. The third adaptive filter 524e processes the second adaptive filter output S512 to generate the third adaptive filter output S516. A fourth adaptive filter 524f (e.g., functionally and/or structurally similar to the fourth adaptive filter 124c of FIG. 1B) receives the third adaptive filter output S516 as an input and the fourth reference signal S518 as a reference input. The fourth adaptive filter 524f processes the third adaptive filter output S516 to generate the fourth adaptive filter output S520. In some instances, the fourth adaptive filter output can have artifacts that result in an output that is not smooth and/or contains noise. As such, a post-processor 524g processes the fourth adaptive filter output S520 to generate the final output S522 which illustrates a smoothed signal. In some embodiments, each of the first, second, third, and fourth adaptive filters can be QR-RLS filters.

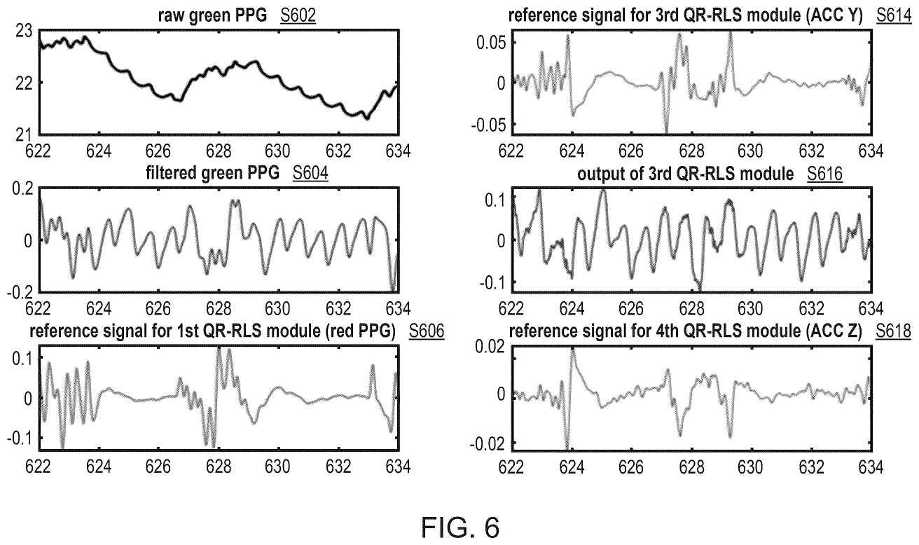

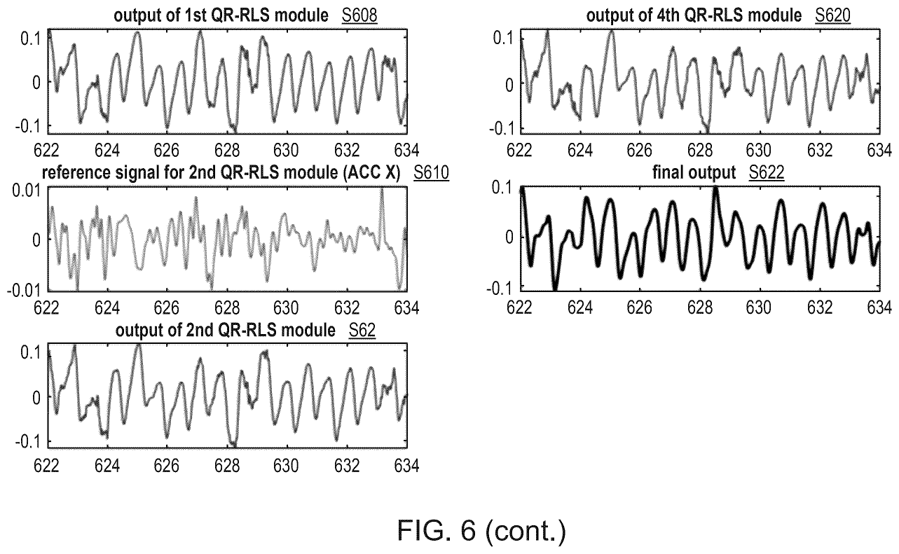

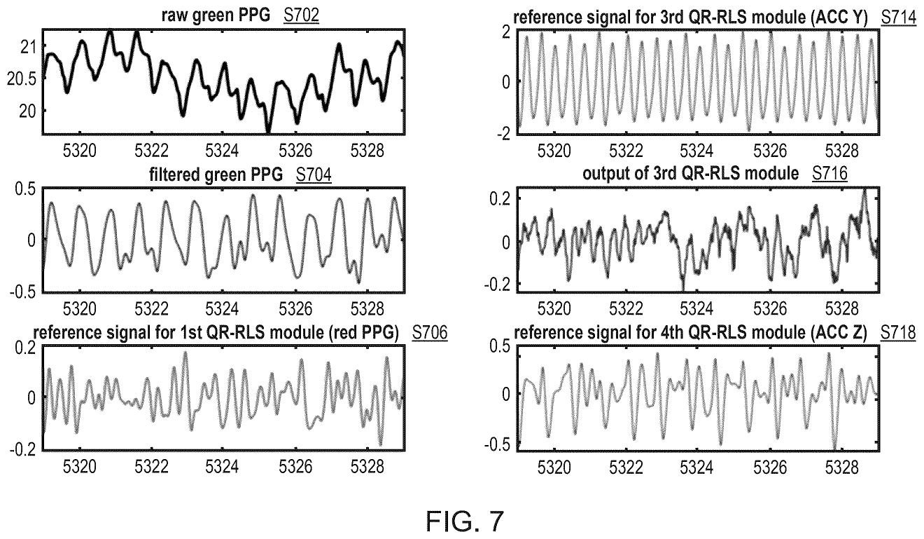

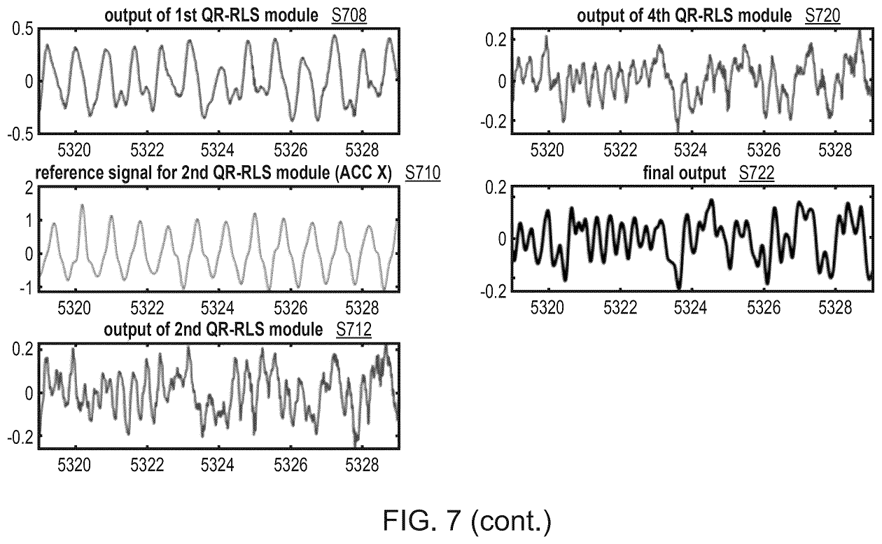

FIGS. 6-7 depict examples of source signals and reference signals, and the processed output at each stage of applying the methods and workflows described herein (e.g., method 400 depicted in FIG. 4, and the workflow depicted in FIG. 5). FIG. 6 corresponds to PPG signals and reference signals acquired when a user is typing on a keyboard, which may predominantly consist of micro-motions. FIG. 7 corresponds to PPG signals and reference signals acquired when a user is running, which may include macro-motions and micro-motions. As shown in the processed outputs in FIGS. 6 and 7, different stages of the MAR algorithms described herein may be better suited for removing artifacts associated with micro-motions and/or macro-motions.

In FIG. 6, a raw green PPG signal S602 can be measured during a set time period, e.g., by a source sensor (e.g., source sensor 116). The raw PPG signal S602 can be filtered by a bandpass filter applying a bandwidth corresponding to 24 beats per minute to 240 beats per minute to produce the filtered green PPG signal S604. A red PPG signal S606 can also be measured during that set time period, e.g., by a reference sensor (e.g., reference sensor 117). The red PPG signal S606 can be used as a reference signal for a 1^{st} QR-RLS filter module (e.g., a first adaptive filter 124c) to process the filtered green PPG signal S604. The output of the 1^{st} QR-RLS filter is shown as signal S608. As shown by that output, most of the motion artifacts (e.g., micro-motions associated with typing) may be canceled out by the 1^{st} QR-RLS filter. An x-axis acceleration signal S610, a y-axis acceleration signal S614, and a z-axis acceleration signal S618 can also be measured during the set time period, e.g., by one or more reference sensors (e.g., reference sensor(s) 117). The x-axis acceleration signal S610 can be used as a reference signal for a 2^{nd} QR-RLS filter module (e.g., a second adaptive filter 124d) to process the output of the 1^{st} QR-RLS filter. The output of the 2^{nd} QR-RLS filter is shown as signal S612. The y-axis acceleration signal S614 can be used as a reference signal for a 3^{rd} QR-RLS filter module (e.g., a third adaptive filter 124e) to process the output of the 2^{nd} QR-RLS filter. The output of the 3^{rd} QR-RLS filter is shown as signal S616. The z-axis acceleration signal S618 can be used as a reference signal for a 4^{th} QR-RLS filter module (e.g., a fourth adaptive filter 124f) to process the output of the 3^{rd} QR-RLS filter. The output of the 4^{th} QR-RLS filter is shown as signal S620. Further post-processing (e.g., smoothing, such as that done by post-processor 524g) can be performed on the output of the 4^{th} QR-RLS filter, which can provide the final output S622.

As shown in FIG. 6, the 2^{nd}, 3^{rd}, and 4^{th} QR-RLS filter modules using accelerometry as references do not significantly alter their respective input signals. This is because the user was largely engaging in micro-movements during typing, and such micro-movements would not have led to significant artifacts that are associated with acceleration in the x-, y-, and/or z-axes.

FIG. 7 provides a similar view as FIG. 6 of the signals being processed by the methods and workflows described herein, except that the artifacts associated with the noise in the raw source signal (e.g., raw green PPG signal) may be removed at a different stage of the MAR algorithm than that shown in FIG. 6. In particular, in FIG. 7, a raw green PPG signal S702 can be measured during a set period when the user is running. The raw PPG signal S702 can be filtered by a bandpass filter applying a bandwidth corresponding to 24 beats per minute to 240 beats per minute to produce the filtered green PPG signal S704. A red PPG signal S706 can also be measured during that set time period. The red PPG signal S706 can be used as a reference signal for a 1^{st} QR-RLS filter module (e.g., a first adaptive filter 124c) to process the filtered green PPG signal S704. The output of the 1^{st} QR-RLS filter is shown as signal S708. As shown by that output, the 1^{st} QR-RLS filter did not cancel out or removal significant motion artifacts from the green PPG signal. This can be due to a lack of correlation between the green PPG signal and the red PPG signal, e.g., due to the user running.

An x-axis acceleration signal S610, a y-axis acceleration signal S714, and a z-axis acceleration signal S718 can also be measured during the set time period. The x-axis acceleration signal S710 can be used as a reference signal for a 2^{nd} QR-RLS filter module (e.g., a second adaptive filter 124d) to process the output of the 1^{st} QR-RLS filter. The output of the 2^{nd} QR-RLS filter is shown as signal S712. The y-axis acceleration signal S714 can be used as a reference signal for a 3^{rd} QR-RLS filter module (e.g., a third adaptive filter 124e) to process the output of the 2^{nd} QR-RLS filter. The output of the 3^{rd} QR-RLS filter is shown as signal S716. The z-axis acceleration signal S718 can be used as a reference signal for a 4^{th} QR-RLS filter module (e.g., a fourth adaptive filter 124f) to process the output of the 3^{rd} QR-RLS filter. The output of the 4^{th} QR-RLS filter is shown as signal S720. Further post-processing (e.g., smoothing, such as that done by post-processor 524g) can be performed on the output of the 4^{th} QR-RLS filter, which can provide the final output S722.

As shown, the 2^{nd} QR-RLS filter module, which uses the x-axis acceleration signal, can clean or remove the majority of the artifacts from the green PPG signal. This can be due to the high correlation between a periodic artifact in the green PPG signal (e.g., as a result of running) and the x-axis acceleration signal. The 3^{rd} and the 4^{th} QR-RLS filter modules do not significantly alter their respective input signals. In the final output S722, the pulsatile signal associated with blood flow in a user's vessel can be seen.

It should be understood that the disclosed embodiments are not intended to be exhaustive, and functional, logical, operational, organizational, structural and/or topological modifications can be made without departing from the scope of the disclosure. As such, all examples and/or embodiments are deemed to be non-limiting throughout this disclosure.

All definitions, as defined and used herein, should be understood to control over dictionary definitions, definitions in documents incorporated by reference, and/or ordinary meanings of the defined terms.

Examples of computer code include, but are not limited to, micro-code or micro-instructions, machine instructions, such as produced by a compiler, code used to produce a web service, and files containing higher-level instructions that are executed by a computer using an interpreter. For example, embodiments can be implemented using Python, Java, JavaScript, C++, and/or other programming languages and development tools. Additional examples of computer code include, but are not limited to, control signals, encrypted code, and compressed code.

The drawings primarily are for illustrative purposes and are not intended to limit the scope of the subject matter described herein. The drawings are not necessarily to scale; in some instances, various aspects of the subject matter disclosed herein can be shown exaggerated or enlarged in the drawings to facilitate an understanding of different features. In the drawings, like reference characters generally refer to like features (e.g., functionally similar and/or structurally similar elements).

The acts performed as part of a disclosed method(s) can be ordered in any suitable way. Accordingly, embodiments can be constructed in which processes or steps are executed in an order different than illustrated, which can include performing some steps or processes simultaneously, even though shown as sequential acts in illustrative embodiments. Put differently, it is to be understood that such features can not necessarily be limited to a particular order of execution, but rather, any number of threads, processes, services, servers, and/or the like that can execute serially, asynchronously, concurrently, in parallel, simultaneously, synchronously, and/or the like in a manner consistent with the disclosure. As such, some of these features can be mutually contradictory, in that they cannot be simultaneously present in a single embodiment. Similarly, some features are applicable to one aspect of the innovations, and inapplicable to others.

Where a range of values is provided, it is understood that each intervening value, to the tenth of the unit of the lower limit unless the context clearly dictates otherwise, between the upper and lower limit of that range and any other stated or intervening value in that stated range is encompassed within the disclosure. That the upper and lower limits of these smaller ranges can independently be included in the smaller ranges is also encompassed within the disclosure, subject to any specifically excluded limit in the stated range. Where the stated range includes one or both of the limits, ranges excluding either or both of those included limits are also included in the disclosure.

The phrase “and/or,” as used herein in the specification and in the embodiments, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements can optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the embodiments, “or” should be understood to have the same meaning as “and/or” as defined above. For example, when separating items in a list, “or” or “and/or” shall be interpreted as being inclusive, i.e., the inclusion of at least one, but also including more than one of a number or list of elements, and, optionally, additional unlisted items. Only terms clearly indicated to the contrary, such as “only one of” or “exactly one of,” or, when used in the embodiments, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the embodiments, shall have its ordinary meaning as used in the field of patent law.

As used herein in the specification and in the embodiments, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements can optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the embodiments, as well as in the specification above, all transitional phrases such as “comprising,” “including,” “carrying,” “having,”“containing,” “involving,”“holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Some embodiments described herein relate to a computer storage product with a non-transitory computer-readable medium (also can be referred to as a non-transitory processor-readable medium) having instructions or computer code thereon for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The media and computer code (also can be referred to as code) can be those designed and constructed for the specific purpose or purposes. Examples of non-transitory computer-readable media include, but are not limited to, magnetic storage media such as hard disks, floppy disks, and magnetic tape; optical storage media such as Compact Disc/Digital Video Discs (CD/DVDs), Compact Disc-Read Only Memories (CD-ROMs), and holographic devices; magneto-optical storage media such as optical disks; carrier wave signal processing modules; and hardware devices that are specially configured to store and execute program code, such as Application-Specific Integrated Circuits (ASICs), Programmable Logic Devices (PLDs), Read-Only Memory (ROM) and Random-Access Memory (RAM) devices. Other embodiments described herein relate to a computer program product, which can include, for example, the instructions and/or computer code discussed herein.

Some embodiments and/or methods described herein can be performed by software (executed on hardware), hardware, or a combination thereof. Hardware modules can include, for example, a processor, a field programmable gate array (FPGA), and/or an application specific integrated circuit (ASIC). Software modules (executed on hardware) can include instructions stored in a memory that is operably coupled to a processor and can be expressed in a variety of software languages (e.g., computer code), including C, C++, Java™, Ruby, Visual Basic™, and/or other object-oriented, procedural, or other programming language and development tools. Examples of computer code include, but are not limited to, micro-code or micro-instructions, machine instructions, such as produced by a compiler, code used to produce a web service, and files containing higher-level instructions that are executed by a computer using an interpreter. For example, embodiments can be implemented using imperative programming languages (e.g., C, Fortran, etc.), functional programming languages (Haskell, Erlang, etc.), logical programming languages (e.g., Prolog), object-oriented programming languages (e.g., Java, C++, etc.) or other suitable programming languages and/or development tools. Additional examples of computer code include, but are not limited to, control signals, encrypted code, and compressed code.

We claim:

1. An apparatus, comprising:
one or more source sensors configured to measure one or more source signals, the one or more source signals including a source signal component and a source noise component;
at least one reference sensor configured to measure at least one reference signal and
a processor operatively coupled to the one or more source sensors and the at least one reference sensor, the processor configured to:
receive the one or more source signals and the at least one reference signal; and
process the one or more source signals using one or more adaptive filters that uses as a reference at least one reference signal from at least one reference sensors to produce a filtered output,
the filtered output having less than or equal to of the source noise component than the one or more source signals.

2. The apparatus of claim 1, wherein the one or more source sensors include at least one a green wavelength photoplethysmography (PPG) sensor, a red PPG sensor, an infrared PPG sensor, an accelerometer, or a gyroscope.

3. The apparatus of claim 1, wherein the at least one reference sensor includes at least one of a red wavelength photoplethysmography (PPG) sensor, a green PPG sensor, an infrared PPG sensor, an accelerometer, or a gyroscope.

4. The apparatus of claim 3, wherein the at least one reference signal includes a PPG signal measured by the red PPG sensor, the green PPG sensor, or the infrared PPG sensor.

5. The apparatus of claim 3, wherein the at least one reference signal includes at least one of an accelerometer signal measured by the accelerometer or a gyroscope signal measured by the gyroscope.

6. The apparatus of claim 1, wherein processing the one or more source signals includes:
process the one or more source signals using a first adaptive filter that uses as a reference a first reference signal from the at least one reference signal to produce a first output; and
process the first output using a second adaptive filter that uses as a reference a second reference signal from the at least one reference signal to produce a second output; and
process the second output using a third adaptive filter that uses as a reference a third reference signal from the at least one reference signal to produce the filtered output.

7. The apparatus of claim 6, wherein the at least one reference sensor includes a 3-axis accelerometer configured to measure an accelerometer signal, and
wherein the first reference signal is a first axis of the accelerometer signal, the second reference signal is a second axis of the accelerometer signal, and the third reference signal is a third axis of the accelerometer signal.

8. The apparatus of claim 1, wherein processing the one or more source signals includes:
process the one or more source signals using a first adaptive filter that uses as a reference a first reference signal from the at least one reference signal to produce a first output; and
process the first output using a second adaptive filter that uses as a reference a second reference signal from the at least one reference signal to produce a second output;
process the second output using a third adaptive filter that uses as a reference a third reference signal from the at least one reference signal to produce a third output; and
process the second output using a fourth adaptive filter that uses as a reference a fourth reference signal from the at least one reference signal to produce the filtered output.

9. The apparatus of claim 8, wherein the one or more reference sensor includes a red wavelength photoplethysmography (PPG) sensor, an infrared PPG sensor, or a green PPG sensor configured to measure a PPG signal and an accelerometer configured to measure an accelerometer signal, and
wherein the first reference signal is the PPG signal, the second reference signal is a first axis of the accelerometer signal, the third reference signal is a second axis of the accelerometer signal, and the fourth reference signal is a third axis of the accelerometer signal.

10. The apparatus of claim 9, wherein the second output has less of the source noise component than the first output, the third output has less of the source noise component than the second output, and the filtered output has less of the source noise component than the third output.

11. The apparatus of claim 1, wherein the one or more of reference sensor includes a gyroscope configured to measure a gyroscopic signal, wherein the at least one reference signals includes at least one of an x-axis gyroscopic signal measured by a gyroscope, a y-axis gyroscopic signal measured by the gyroscope, or a z-axis gyroscopic signal measured by the gyroscope.

12. A method, comprising:
receiving one or more source signals measured by one or more source sensors;
receiving a plurality of reference signals measured by a plurality of reference sensors, the plurality of reference sensors including at least two different types of sensors; and
processing the one or more source signals using one or more adaptive filters that uses as a reference at least one reference signal from the plurality of reference signals to produce a filtered output;
the filtered output having less than or equal to of the source noise component than the one or more source signals.

13. The method of claim 12, wherein the one or more source signals are not saturated.

14. The method of claim 12, further comprising:
compensating for a saturation associated with the one or more source signals.

15. The method of claim 14, wherein compensating for the saturation includes adjusting the operation of the one or more source sensors.

16. The method of claim 14, wherein compensating for the saturation includes using at least one of adjusting a gain, using a compensation voltage, using a compensating current, or using a compensation algorithm.

17. A method, comprising:
receiving one or more source signals measured by one or more source sensor;
receiving a plurality of reference signals measured by at least one sensor associated with a user and at least one sensor configured to measure movement, the plurality of reference sensors including at least two different types of sensors; and
processing the one or more source signals using a first adaptive filter that uses as a reference at least one reference signal from the at least one sensor associated with the user to produce a first output;
processing the first output using one or more adaptive filters that uses as a reference at least one reference signal from the at least one sensor configured to measure movement to produce a filtered output;
the filtered output having less than or equal to of the source noise component than the one or more source signals.

18. The method of claim 17, wherein the at least one sensor associated with the user includes at least one of a red wavelength photoplethysmography (PPG) sensor, a green PPG sensor, or an infrared PPG sensor.

19. The method of claim 17, wherein the at least one sensor configured to measure movement includes an accelerometer.

20. The method of claim 17, wherein the at least one sensor configured to measure movement includes a gyroscope.

21. The method of claim 17, wherein at least one of the one or more source sensors or the plurality of reference sensors operates intermittently.
